(19) United States
(12) Patent Application Publication
(10) Pub. No.: US 2024/0366095 A1
(43) Pub. Date: Nov. 7, 2024

(54) TECHNOLOGIES FOR MULTIMODAL SENSOR WEARABLE DEVICE FOR BIOMEDICAL MONITORING

(71) Applicant: THE TEXAS A&M UNIVERSITY SYSTEM, College Station, TX (US)

(72) Inventors: Gerard L. COTE, College Station, TX (US); Justin MCMURRAY, College Station, TX (US); Kimberly BRANAN, College Station, TX (US); Myeong NAMKOONG, College Station, TX (US); Limei TIAN, College Station, TX (US); Sung II PARK, College Station, TX (US)

(21) Appl. No.: 18/561,132

(22) PCT Filed: May 23, 2022
(86) PCT No.: PCT/US2022/030486

§ 371 Date: Nov. 15, 2023

Related U.S. Application Data
(60) Provisional application No. 63/191,402, filed on May 21, 2021.

(51) Int. Cl. A61B 5/021; A61B 5/024; A61B 5/0531

(52) U.S. Cl. CPC A61B 5/02108; A61B 5/02141; A61B 5/02433; A61B 5/0531; A61B 2560/0247; A61B 2560/0252; A61B 2562/0219; A61B 2562/164

(57) ABSTRACT

Technologies for multimodal sensing include a wearable device having a flexible substrate and at least one multimodal sensor coupled to the flexible substrate. The multimodal sensor includes a photoplethysmography sensor and a bioimpedance sensor. The wearable device may include context sensors such as an accelerometer, a gyroscope, a temperature sensor, or a pressure sensor. The multimodal sensor may include an integrated pressure sensor. A processor of the wearable device is configured to receive sensor data from the photoplethysmography sensor and the bioimpedance sensor of the multimodal sensor that is indicative of a pulse curve of a user. The processor is further configured to correct the sensor data based on data received from the context sensors and to determine blood pressure data based on the corrected sensor data. The processor may be configured to store the blood pressure data or to transmit the blood pressure data to a remote computing device.

### CROSS-REFERENCE TO RELATED APPLICATIONS

This application claims the benefit of and priority to U.S. Patent Application No. 63/191,402, entitled “MULTI-MODAL WEARABLE DEVICE FOR BIOMEDICAL MONITORING,” which was filed on May 21, 2021.

### STATEMENT REGARDING FEDERALLY SPONSORED RESEARCH

This invention was made with Government support under contract number 1648451, awarded by the National Science Foundation. The Government has certain rights in this invention.

### BACKGROUND

The development of digital tools and devices for the health care and fitness industries is growing exponentially. However, the majority of new innovations in this area concern wellness applications for relatively healthy, middle- to upper-middle class populations, and do not directly measure diagnostic markers or disease symptoms. Additionally, current technologies are subject to motion artifact, biodiversity, and other noise sources and thus may lack accuracy. For example, typical systems may use photoplethysmography (PPG) sensors in combination with electrocardiogram (ECG) data to measure pulse transit time (PTT) and pulse wave velocity (PWV). However, using ECG/PPG measurement may have limited accuracy, because ejection fraction time is variable within and between patients. Additionally, ECG/PPG systems typically measure mean arterial pressure and thus provide only approximations for systolic or diastolic values. Furthermore, current devices are often expensive and cumbersome to use. For example, typical oscillometric devices require the use of inflatable cuffs, which are uncomfortable and require time for inflation/deflation and recovery between measurements, and thus are capable only of discrete measurements. Moreover, most current technologies in the fitness tracker space are unregulated or otherwise not regulator-approved, and thus may not be useful for making true health decisions.

### SUMMARY

According to one aspect of the disclosure, a wearable device includes a flexible substrate and a multimodal sensor coupled to the flexible substrate. The multimodal sensor comprises a photoplethysmography sensor and a bioimpedance sensor. In an embodiment, the wearable device further includes a processor coupled to the flexible substrate, wherein the processor is configured to receive sensor data from the multimodal sensor.

In an embodiment, the bioimpedance sensor comprises an electrode positioned on an external surface of the wearable device. In an embodiment, the photoplethysmography sensor comprises a light-emitting diode (LED) and a photodiode. In an embodiment, the LED comprises a near-infrared LED. In an embodiment, the LED and the photodiode are positioned adjacent to the electrode on the external surface of the wearable device.

In an embodiment, the wearable device further includes a second sensor configured to generate sensor data indicative of an environment of the wearable device. In an embodiment, the second sensor comprises a temperature sensor or a pressure sensor. In an embodiment, the wearable device further comprises a second sensor configured to generate sensor data indicative of motion of the wearable device. In an embodiment, the second sensor comprises an accelerometer or a gyroscope.

In an embodiment, the wearable device further includes a wireless communication circuit.

In an embodiment, the flexible substrate comprises a first side and a second side opposite the first side. The multimodal sensor is positioned on the first side of the wearable device. The wearable device further comprises an adhesive coating coupled to the first side, wherein the adhesive coating surrounds the multimodal sensor.

In an embodiment, the wearable device further includes a processor configured to receive sensor data from the photoplethysmography sensor of the multimodal sensor and the bioimpedance sensor of the multimodal sensor. In an embodiment, the wearable device further includes a flexible conductive path coupled to the flexible substrate, wherein the flexible conductive path electrically couples the processor to the multimodal sensor. In an embodiment, the processor is further configured to generate biometric data based on the sensor data received from the photoplethysmography sensor and from the bioimpedance sensor, wherein the biometric data comprises blood pressure data.

In an embodiment, the multimodal sensor further comprises an integrated pressure sensor. In an embodiment, the multimodal sensor comprises a first sensor frame and a second sensor frame. The first sensor frame is coupled to the flexible substrate, the integrated pressure sensor is coupled between the first sensor frame and the second sensor frame, and the photoplethysmography sensor and the bioimpedance sensor are coupled to the second sensor frame.

In an embodiment, the flexible substrate comprises a stretchable composite. In an embodiment, the flexible substrate comprises a breathable composite.

According to another aspect, a wearable device for continuous cuffless blood pressure monitoring includes a multimodal sensor device comprising a photoplethysmography sensor and a bioimpedance sensor, a sensor manager, and a biometric analyzer. The sensor manager is to receive first sensor data from the photoplethysmography sensor of the multimodal sensor device, wherein the first sensor data is indicative of a pulse curve of a user, and to receive second sensor data from the bioimpedance sensor of the multimodal sensor device, wherein the second sensor data is indicative of the pulse curve of the user. The biometric analyzer is to determine blood pressure data based on the first sensor data and the second sensor data.

In an embodiment, the wearable device further includes a context sensor and a signal conditioner. The sensor manager is further to receive third sensor data from the context sensor of the wearable device. The signal conditioner is to correct the first sensor data and the second sensor data based on the third sensor data to generate corrected sensor data. To determine the blood pressure data comprises to determine the blood pressure data based on the corrected sensor data. In an embodiment, to receive the third sensor data comprises to receive motion sensor data from an accelerometer or a gyroscope of the wearable device. In an embodiment, to correct the first sensor data and the second sensor data comprises to remove motion artifacts from the first sensor data and the second data based on the third sensor data. In an embodiment, to receive the third sensor data comprises to receive temperature data from a temperature sensor of the wearable device. In an embodiment, to receive the third sensor data comprises to receive pressure data from a pressure sensor of the wearable device. In an embodiment, the pressure sensor is integrated in the multimodal sensor device.

In an embodiment, to determine the blood pressure data comprises determine a pulse transit time. In an embodiment, to determine the blood pressure data comprises to determine a pulse wave velocity. In an embodiment, to determine the pulse wave velocity comprises to receive third sensor data from a second photoplethysmography sensor of a second multimodal sensor device, wherein the second multimodal sensor device is positioned on the wearable device a first distance from the multimodal sensor device; receive fourth sensor data from a second bioimpedance sensor of the second multimodal sensor device; determine a difference in pulse arrival time by a comparison of the first sensor data and the second sensor data to the third sensor data and the fourth sensor data; and determine the pulse wave velocity based on the difference in pulse arrival time and the first distance.

In an embodiment, to receive the first sensor data and to receive the second sensor data comprises to receive the first sensor data and the second sensor data at a sampling rate of at least about 10 KHz.

In an embodiment, to determine the blood pressure data further comprises to determine an additional feature of the pulse curve based on the first sensor data and the second sensor data. The additional feature comprises a change in curve shape, an amplitude, a frequency, a slope, an area under the curve, a key point on the curve, or a derivative.

In an embodiment, the biometric analyzer is further to transmit the blood pressure data to a remote computing device. In an embodiment, the biometric analyzer is further to store the blood pressure data with a data storage device.

According to another aspect, a method for continuous cuffless blood pressure monitoring includes receiving, by a processor, first sensor data from a photoplethysmography sensor of a multimodal sensor device, wherein the multimodal sensor device is included in a wearable device, and wherein the first sensor data is indicative of a pulse curve of a user; receiving, by the processor, second sensor data from a bioimpedance sensor of the multimodal sensor device, wherein the second sensor data is indicative of the pulse curve of the user; and determining, by the processor, blood pressure data based on the first sensor data and the second sensor data.

In an embodiment, the method further includes receiving, by the processor, third sensor data from a context sensor of the wearable device; and correcting, by the processor, the first sensor data and the second sensor data based on the third sensor data to generate corrected sensor data; wherein determining the blood pressure data comprises determining the blood pressure data based on the corrected sensor data. In an embodiment, receiving the third sensor data comprises receiving motion sensor data from an accelerometer or a gyroscope of the wearable device. In an embodiment, correcting the first sensor data and the second sensor data comprises removing motion artifacts from the first sensor data and the second data based on the third sensor data. In an embodiment, receiving the third sensor data comprises receiving temperature data from a temperature sensor of the wearable device. In an embodiment, receiving the third sensor data comprises receiving pressure data from a pressure sensor of the wearable device. In an embodiment, the pressure sensor is integrated in the multimodal sensor device.

In an embodiment, determining the blood pressure data comprises determining a pulse transit time. In an embodiment, determining the blood pressure data comprises determining a pulse wave velocity. In an embodiment, determining the pulse wave velocity comprises receiving, by the processor, third sensor data from a second photoplethysmography sensor of a second multimodal sensor device, wherein the second multimodal sensor device is positioned on the wearable device a first distance from the multimodal sensor device; receiving, by the processor, fourth sensor data from a second bioimpedance sensor of the second multimodal sensor device; determining, by the processor, a difference in pulse arrival time by comparing the first sensor data and the second sensor data to the third sensor data and the fourth sensor data; and determining, by the processor, the pulse wave velocity based on the difference in pulse arrival time and the first distance.

In an embodiment, receiving the first sensor data and receiving the second sensor data comprises receiving the first sensor data and the second sensor data at a sampling rate of at least about 10 KHz.

In an embodiment, wherein determining the blood pressure data further comprises determining an additional feature of the pulse curve based on the first sensor data and the second sensor data, wherein the additional feature comprises a change in curve shape, an amplitude, a frequency, a slope, an area under the curve, a key point on the curve, or a derivative.

In an embodiment, the method further includes transmitting, by the processor, the blood pressure data to a remote computing device. In an embodiment, the method further includes storing, by the processor, the blood pressure data with a data storage device.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a wearable device for biometric monitoring with multimodal sensors;

FIG. 2 is a top view of at least one embodiment of the wearable device of FIG. 1;

FIG. 3 is a bottom view of at least one embodiment of the wearable device of FIG. 2;

FIG. 4 is a side view of at least one embodiment of the wearable device of FIGS. 2-3;

FIG. 5 is a schematic diagram illustrating the wearable device of FIGS. 2-4 in use;

FIG. 6 is a bottom view of at least one embodiment of a multimodal sensor device with an integrated pressure sensor;

FIG. 7 is an exploded view of the multimodal sensor device of FIG. 6;

FIG. 8 is a perspective view of at least one embodiment of the wearable device of FIG. 1;

FIG. 9 is an exploded view of the wearable device of FIG. 8;

FIG. 10 is a simplified block diagram of at least one embodiment of an environment that may be established by a processor of the wearable device of FIG. 1; and

FIG. 11 is a simplified flow diagram of at least one embodiment of a method for biometric monitoring with multimodal sensors that may be executed by the wearable device of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C): (A and B); (B and C); or (A, B, and C). Similarly, items listed in the form of “at least one of A, B, or C” can mean (A); (B); (C): (A and B); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

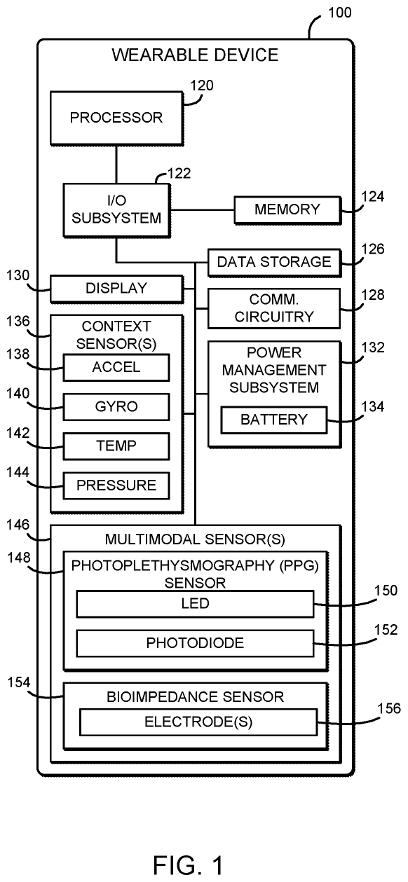

Referring now to FIG. 1, an illustrative wearable device 100 for biometric monitoring includes one or more multimodal sensors 146 coupled to a processor 120. Each multimodal sensor 146 includes multiple sensor devices including, illustratively, at least one photoplethysmography (PPG) sensor 148 and at least one bioimpedance sensor 154. In use, as described further below, the wearable device 100 is secured to a user's skin, for example by being positioned on the user's inner forearm, over the radial artery. The processor 120 collects sensor data from both of the PPG sensors 148 and the bioimpedance sensors 154 of the multimodal sensors 146, and processes that sensor data to generate continual blood pressure monitoring data or other biometric data. By generating blood pressure data based on sensor data collected using multiple anatomical mechanisms located at the same position on the user, the wearable device 100 may reduce noise, increase accuracy, and increase local anatomical resolution as compared to conventional blood pressure monitoring systems. Additionally, by collecting multimodal sensor data from the multimodal sensors 146, the wearable device 100 may overcome variability in ejection fraction period that is inherent in conventional ECG/PPG based signals. In some embodiments, the processor 120 may also collect sensor data from one or more additional, contextual sensors 136 and use that sensor data to remove motion artifacts or otherwise correct the sensor data collected from the multimodal sensor 146. By correcting the sensor data based on motion data or other contextual data, the wearable device 100 may improve accuracy as compared to typical systems.

Additionally, the wearable device 100 may be embodied as a lightweight, breathable device capable of measuring blood pressure without requiring a traditional inflatable cuff (e.g., a forearm cuff, wrist cuff, or finger cuff). Thus, the wearable device 100 may be capable of continuous blood pressure monitoring throughout the day, which may improve measurement accuracy and enable improved medical care. For example, continuous blood pressure monitoring may be important in determining biomarkers that can reveal stages of cardiovascular disease along with other underlying, damaging hypertensive ailments. Such continuous blood pressure monitoring may enable more comprehensive assessment of blood pressure status and treatment adherence. Accordingly, in some embodiments, the wearable device 100 may be capable of sufficient accuracy for regulatory approval as a medical device, for example by performing cuffless blood pressure monitoring with a tolerance of ±4 mmHg when measuring systolic and diastolic blood pressure.

The illustrative wearable device 100 may be embodied as, without limitation, a flexible patch with integrated sensors, a medical device, a consumer electronics device, a fitness tracker, a smartwatch, a mobile computing device, a multiprocessor system, or any other wearable device capable of performing the functions described herein. As shown in FIG. 1, the illustrative wearable device 100 includes a processor 120, an I/O subsystem 122, memory 124, a data storage device 126, and communication circuitry 128. Of course, the wearable device 100 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124 and/or the data storage 126, or portions thereof, may be incorporated in the processor 120 in some embodiments.

The processor 120 may be embodied as any type of processor or compute engine capable of performing the functions described herein. For example, the processor may be embodied as a microcontroller, digital signal processor, single or multi-core processor(s), or other processor or processing/controlling circuit. Similarly, the memory 124 may be embodied as any type of volatile or non-volatile memory or data storage capable of performing the functions described herein. In operation, the memory 124 may store various data and software used during operation of the wearable device 100 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the wearable device 100. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the wearable device 100, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication circuitry 128 of the wearable device 100 may be embodied as any communication circuit, device, or collection thereof, capable of enabling communications between the wearable device 100 and remote devices. The communication circuitry 128 may be configured to use any one or more communication technology (e.g., wireless or wired communications) and associated protocols (e.g., Ethernet, Bluetooth®, Bluetooth Low Energy (BLE), Wi-Fi®, WiMAX, etc.) to effect such communication.

As shown in FIG. 1, in some embodiments, the wearable device 100 may include a display 130. The display 130 may be embodied as any type of display capable of displaying digital images or other information, such as a liquid crystal display (LCD), a light emitting diode (LED), a plasma display, a cathode ray tube (CRT), or other type of display device. As described further below, the display 130 may be used to display sensor data, biometrics data, or other data generated by the processor 120.

The wearable device 100 further includes a power management subsystem 132 and a power storage device such as a battery 134. The power management subsystem 132 distributes power to the components of the wearable device 100, manages charging the battery 134, and performs other power management functions for the wearable device 100. Additionally or alternatively, although illustrated as a rechargeable lithium-ion battery 134, in some embodiments the battery 134 may be replaceable or otherwise non-rechargeable.

The wearable device 100 may also include one or more context sensors 136, such as an accelerometer 138, a gyroscope 140, a temperature sensor 142, and/or a pressure sensor 144. The accelerometer 138 may be embodied as digital sensor capable of generating sensor data indicative of acceleration of the wearable device 100, such as a 3-axis digital accelerometer. Similarly, the gyroscope 140 may be embodied as a digital sensor capable of measuring orientation and angular velocity of the wearable device 100. In some embodiments, wearable device 100 may include a combined motion sensor such as a 6-axis MEMS motion sensor that performs the functions of the accelerometer 138 and the gyroscope 140. The temperature sensor 142 may be embodied as a digital temperature sensor (DTS), digital thermometer, or other device capable of measuring the temperature of the wearable device 100. The pressure sensor 144 may be embodied as a piezoelectric pressure transducer, a ceramic pressure transducer, a strain gauge, or any other sensor capable of measuring pressure exerted on the wearable device 100.

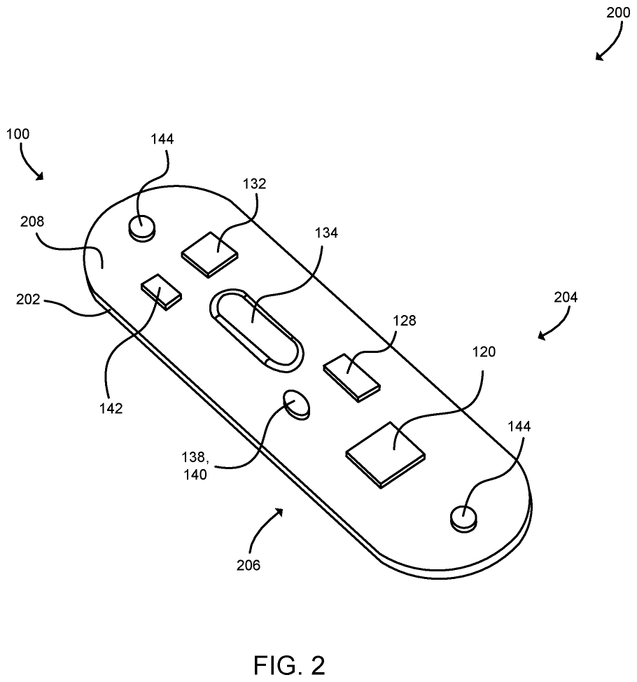

The wearable device 100 further includes one or more multimodal sensors 146. Each multimodal sensor 146 integrates multiple sensors for measuring blood pressure data or other biometrics of the user into a single sensor device. Each sensor may be optimized for maximum signal, low noise, small form factor, and low power consumption. Illustratively, the multimodal sensor 146 includes a photoplethysmography (PPG) sensor 148 and a bioimpedance sensor 154. As illustrated below in connection with FIGS. 2-4 and 6-7, various sensing components of the PPG sensor 148 may be positioned adjacent to, surrounded by, or otherwise in proximity to sensing components of the bioimpedance sensor 154.

The PPG sensor 148 may be embodied as a sensor device that uses changes in light intensity to measure changes in blood volume of the user. Illustratively, the PPG sensor 148 includes a light emitting diode (LED) transmitter 150 and a photodiode receiver 152. In use, the LED 150, which may be embodied as a near-infrared LED, emits light toward the user's body. The photodiode 152 receives light transmitted and/or reflected through the user's skin and underlying blood vessels. The amount of light received by the photodiode 152 varies with the volume of blood within the user's blood vessels and thus measures the user's pulse.

The bioimpedance sensor 154 may be embodied as a sensor device that measures changes in electrical impedance of part of the user's body. Illustratively, the bioimpedance sensor 154 includes an electrode 156 that is positioned in contact with the user's skin. In use, the bioimpedance sensor 154 measures changes in electrical impedance due to changes in cellular shapes and orientations, which occur as a pulse wave moves through the arteries adjacent to the cells. Accordingly, the measured bioimpedance also measures the user's pulse. As described further below, the electrode 156 may surround or otherwise be positioned in proximity to the LED 150 and the photodiode 152, which allows the multimodal sensor 146 to measure the same position of the patient's body using multiple sensing modalities.

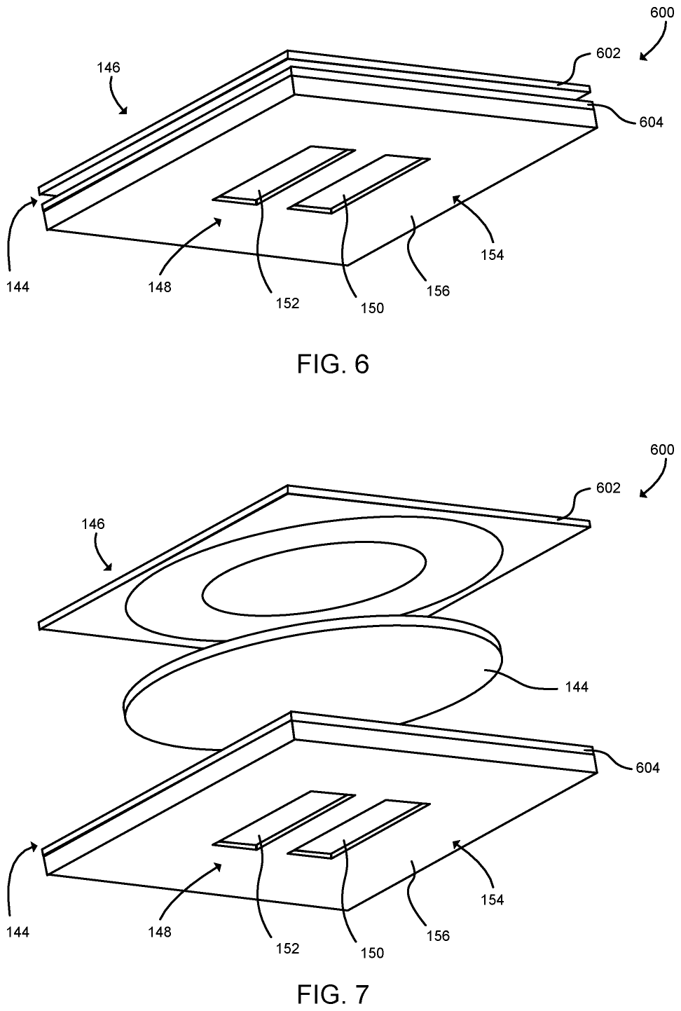

In addition to the PPG sensor 148 and the bioimpedance sensor 154, in some embodiments the multimodal sensor 146 may include additional sensors. For example, as shown in FIGS. 6-7 and discussed further below, in some embodiments the multimodal sensor 146 may include an integrated pressure sensor 144.

Referring now to FIGS. 2-4, one embodiment of a wearable device 200 is shown. As described further below, the wearable device is a flexible patch with an adhesive that may be attached to the user's skin in order to perform continuous, cuffless blood pressure monitoring. The wearable device 200 includes a flexible substrate 202. The flexible substrate 202 may be embodied as a stretchable, breathable composite material that is suitable for wearing on the user's skin for extended periods of time. The flexible substrate 202 has a top side 204 and a bottom side 206.

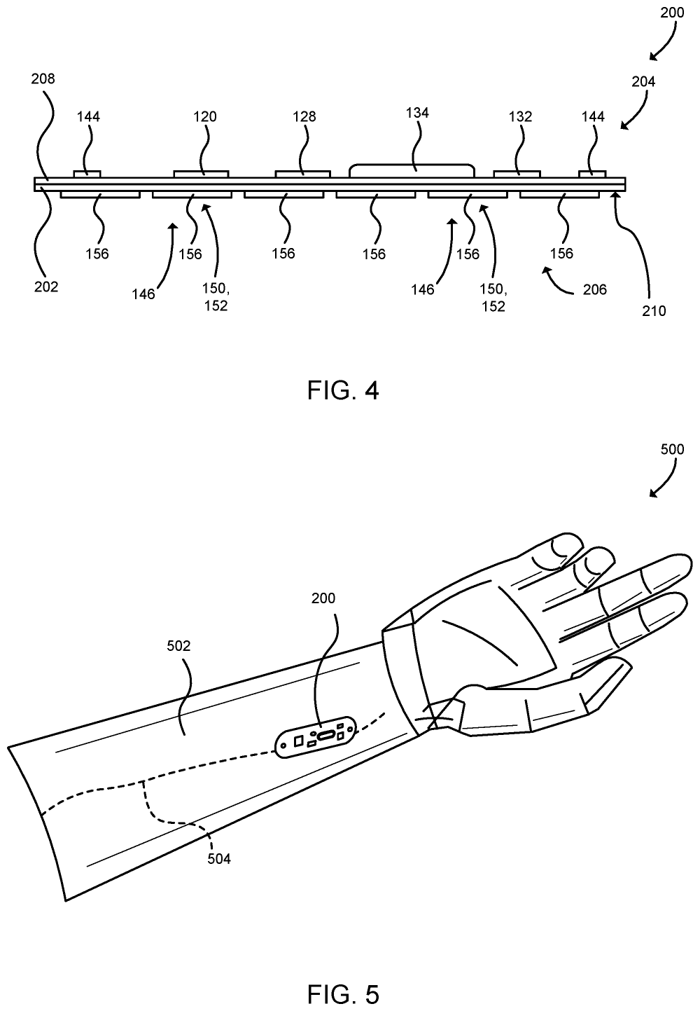

As shown in FIGS. 2 and 4, multiple components of the wearable device 200 are positioned on the top side 204 of the wearable device 200. Illustratively, the top side 204 includes a pair of pressure sensors 144, a power management subsystem 132, which is illustratively a power management integrated circuit (PMIC), a temperature sensor 142, a battery 134, a combined accelerometer 138 and gyroscope 140, a communications circuit 128, which is illustratively a Bluetooth Low Energy (BLE) module, and a processor 120, which is illustratively a microcontroller. The top side 204 further includes an electrical ground plane 208 that connects together those components of the wearable device 200. As discussed above, the flexible substrate 202 may be stretchable or otherwise flexible, but the various components positioned on the flexible substrate 202, such as the processor 120, the context sensors 136, the multimodal sensors 146, and other components, may be rigid. Those components may be connected by one or more flexible traces, flexible wiring, bio-ink, or other flexible electronic components.

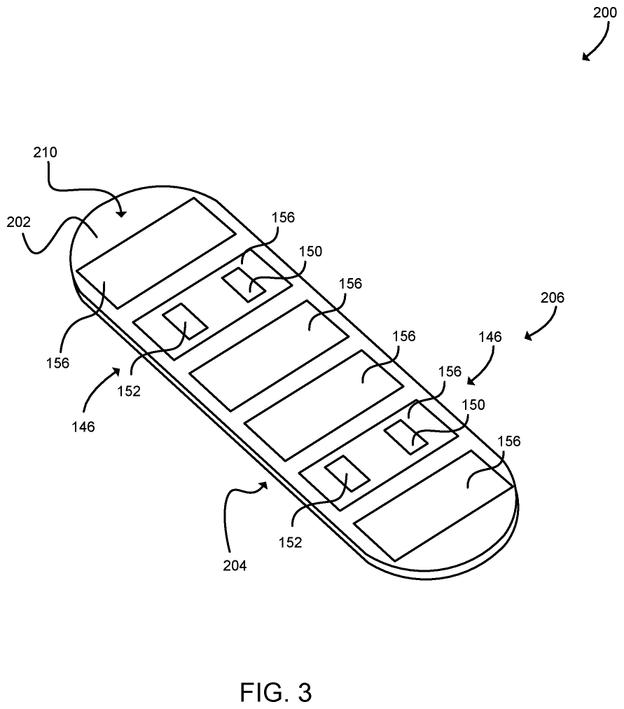

As shown in FIGS. 3 and 4, multiple multimodal sensors 146 are positioned on the bottom side 206 of the wearable device 200. Each multimodal sensor 146 includes an electrode 156 positioned on the bottom side 204. Additionally, each multimodal sensor 146 include an LED 150 and photodiode 152 positioned on the bottom side 206. Each of the LED 150 and the photodiode 152 is surrounded by an electrode 156. As discussed above, this allows the PPG sensor 148 and the bioimpedance sensor 154 to measure the pulse wave at the same position on the user's body. The bottom side 206 further includes an adhesive coating 210 surrounding the multimodal sensors 146. The adhesive 210 is configured to attach to the user's skin in order to secure the wearable device 200 to the user.

In use, the bottom side 206 of the wearable device 200 is placed in contact with the user's skin, and the adhesive 210 secures the wearable device to the skin. When the wearable device 200 is positioned on the skin, the electrodes 156 of the multimodal sensors 146 are in direct contact with the user's skin. Similar, when the wearable device 200 is positioned on the skin, the LEDs 150 are positioned to shine light into the user's body, and the photodiodes 152 are positioned to measure reflected and/or transmitted light. The pressure sensors 144 may measure how tightly the wearable device 200 is held against the body, which may be used to correct multimodal sensor 146 measurements as described further below.

Referring now to FIG. 5, schematic diagram 500 illustrates an arm 502 of the user. In some embodiments, as shown, the wearable device 200 may be adhered to the inner part of the arm 502, above a radial artery 504. In this position, the wearable device 200 may collect accurate blood pressure data. Additionally, the sensors 146 are stabilized on the skin by the flexible substrate 202, which may reduce noise in the measurement data. In this position, the wearable device 200 is unobtrusive and maintains a low profile and, thus, may reduce the likelihood of accidental contact between the wearable device 200 and external objects. Additionally or alternatively, although illustrated as being positioned over the radial artery 504, in other embodiments the wearable device 200 may be positioned on a different part of the patient's body, such as on a different artery, on an arteriole, or on an area with no arteries.

Referring now to FIGS. 6-7, one potential embodiment of a multimodal sensor 600 including an integrated pressure sensor 144 is shown. As shown, the multimodal sensor 600 includes a PPG sensor 148 and a bioimpedance sensor 154. The electrode 156 of the bioimpedance sensor 154 is positioned on a bottom surface of the multimodal sensor 600. The LED 150 and photodiode 152 of the PPG sensor 148 are also positioned on the bottom surface of the multimodal sensor 600, surrounded by the electrode 156. The multimodal sensor 600 further includes an upper sensor receiver 602 and a lower sensor receiver 604. As shown in FIG. 7, the pressure sensor 144 is positioned between the upper sensor receiver 602 and the lower sensor receiver 604. The pressure sensor 144 is illustratively a piezoelectric pressure sensor; however, as described above, in other embodiments the pressure sensor 144 may be a ceramic pressure sensor or any other appropriate pressure sensing device. The upper sensor receiver 602 may be attached to a wearable device 100, 200, for example to the flexible substrate 202 of the wearable device 200 as shown in FIGS. 2-4. In use, when the multimodal sensor 600 is pressed against the user's skin, the pressure sensor 144 is compressed between the upper sensor receiver 602 and the lower sensor receiver 604. The pressure sensor 144 may thus measure pressure exerted on the multimodal sensor 600.

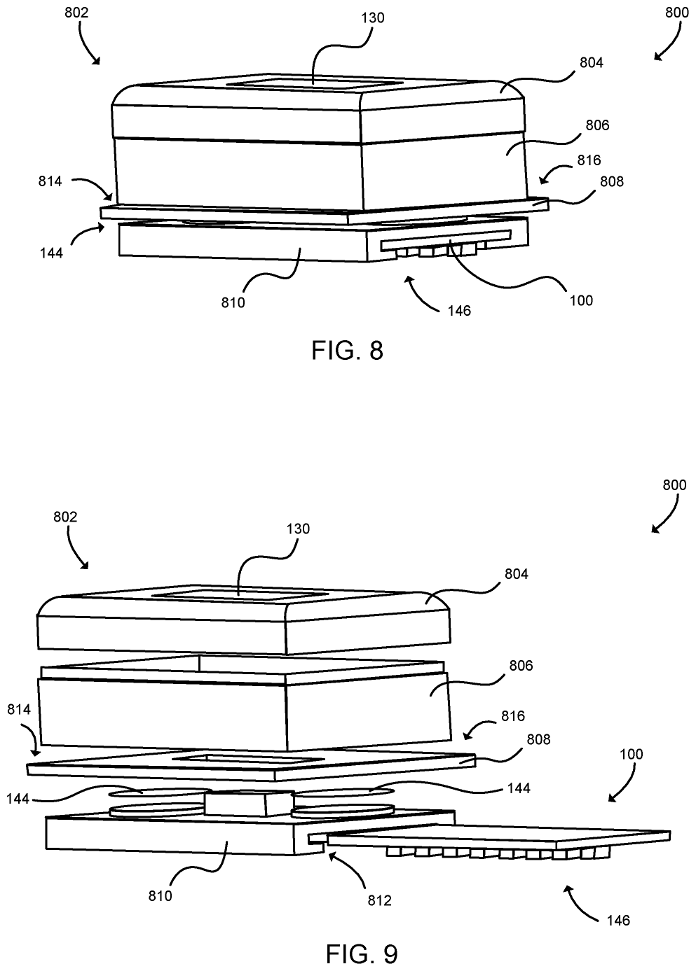

Referring now to FIGS. 8-9, another illustrative embodiment of a wearable device 800 is shown. The illustrative wearable device 800 includes an outer casing 802 that receives a wearable device 100, which may be embodied as a printed circuit board (PCB) including components as shown in FIG. 1, a flexible substrate as shown in FIGS. 2-4, or another embodiment of the wearable device 100. The casing 802 is assembled from a display cover 804, a microcontroller case 806, an upper sensor receiver 808, and a lower sensor receiver 810. As shown in FIG. 9, the wearable device 100 slides into a slot 812 formed in the lower sensor receiver 810. A pair of pressure sensors 144 are positioned between the lower sensor receiver 810 and the upper sensor receiver 808. The display cover 804 includes the display 130, which may be embodied as an OLED display or other display capable of displaying pressure data from the pressure sensors 144, blood pressure data, or other data generated by the wearable device 100. When assembled, as shown in FIG. 8, the multimodal sensors 146 are exposed through the bottom of the lower sensor receiver 810. Thus, the wearable device may be placed in contact with the user's skin in order to perform continuous blood pressure monitoring measurements. In some embodiments, the wearable device 800 may be secured to the user using one or more straps or other devices. For example, in some embodiments, an adjustable fabric strap may be connected to extensions 814, 816 on either side of the upper sensor receiver 808, allowing the wearable device 800 to be strapped to the user's arm or other location on the user's body. Additionally, tension applied to the upper sensor receive 808 by such a strap may be adjusted, using pressure sensor 144 feedback shown on the display 130, in order to achieved a desired fit on the user.

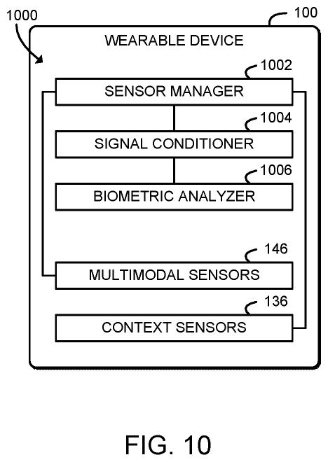

Referring now to FIG. 10, in the illustrative embodiment, the wearable device 100 establishes an environment 1000 during operation. The illustrative environment 1000 includes a sensor manager 1002, a signal conditioner 1004, and a biometric analyzer 1006. The various components of the environment 1000 may be embodied as hardware, firmware, software, or a combination thereof. As such, in some embodiments, one or more of the components of the environment 1000 may be embodied as circuitry or a collection of electrical devices (e.g., sensor manager circuitry 1002, signal conditioner circuitry 1004, and/or biometric analyzer circuitry 1006). It should be appreciated that, in such embodiments, one or more of those components may form a portion of the processor 120, the multimodal sensor 146, the context sensor 136, and/or other components of the wearable device 100.

The sensor manager 1002 is configured to receive sensor data from one or more multimodal sensors 146, including sensor data from the photoplethysmography sensor 148 and the bioimpedance sensor 152 of each multimodal sensor device 146. The first sensor data is indicative of a pulse curve of a user. The sensor data may be received at a sampling rate of at least about 10 kHz. The sensor manager 1002 may be further configured to receive sensor data from one or more context sensors 136 of the wearable device 100, such as motion data from an accelerometer 138 or a gyroscope 140, temperature data from a temperature sensor 142, or pressure data from a pressure sensor 144.

The signal conditioner 1004 is configured to correct the sensor data received from the multimodal sensors 146 based on the sensor data received from the context sensors 136 to generate corrected sensor data. Correcting the sensor data may include removing motion artifacts from the sensor data based on motion data received from the accelerometer 138 or the gyroscope 140.

The biometric analyzer 1006 is configured to determine blood pressure data based on the sensor data received from the multimodal sensors 146 as corrected based on the sensor data received from the context sensors 136. Determining the blood pressure data may include determining a pulse transit time or a pulse wave velocity. Determining the pulse wave velocity may include determining a difference in pulse arrival time by comparing of sensor data received from multiple multimodal sensors 146 that are separated by a particular distance and then determining the pulse wave velocity based on the difference in pulse arrival time and the distance. In some embodiments determining the blood pressure data may include determining an additional feature of the pulse curve based on the sensor data from the multimodal sensors 146. The additional feature may include a change in curve shape, an amplitude, a frequency, a slope, an area under the curve, a key point on the curve, or a derivative. The biometric analyzer 1006 may be further configured to transmit the blood pressure data to a remote computing device or to store the blood pressure data with a data storage device.

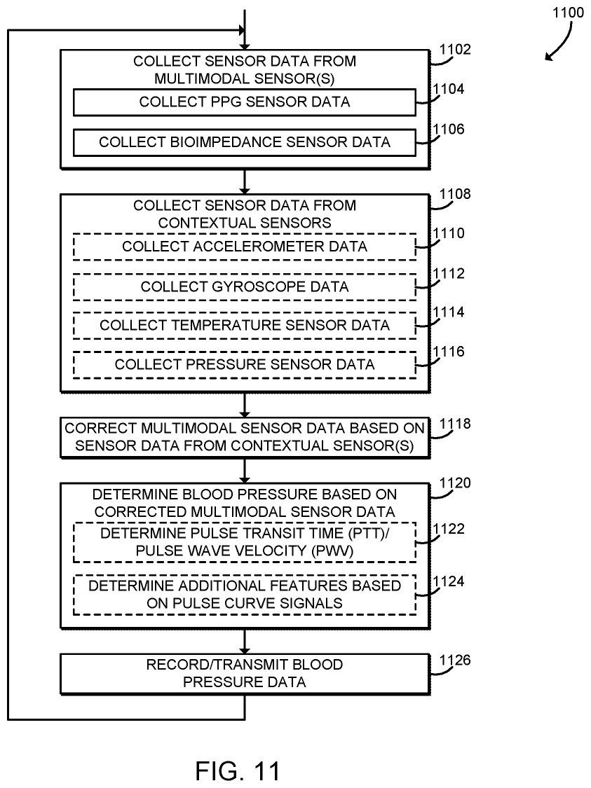

Referring now to FIG. 11, in use, the processor 120 may execute a method 1100 for biometric monitoring with multimodal sensors. It should be appreciated that, in some embodiments, the operations of the method 1100 may be performed by one or more components of the environment 1000 of the controller wearable device 100 as shown in FIG. 10. The method 1100 begins with block 1102, in which the processor 120 collects sensor data from one or more multimodal sensors 146 of the wearable device 100. As described further below, in order to enable measurement of pulse wave velocity (PWV), the processor 120 may sample sensor data at a sample rate of greater than about 10 kHz. Thus, the processor 120 may be capable of measuring PWV using multimodal sensors 146 separated by a few centimeters, as shown in FIGS. 2-5.

In block 1104 the processor 120 collects sensor data from the PPG sensors 148. As described above, during measurement the LED 150 of each PPG sensor 148 shines light onto the user's body, and the photodiode 152 of each PPG sensor 148 measures transmitted or reflected light. The amplitude of this measured light changes with the amount of blood volume. Thus, the sensor data collected by the PPG sensor 148 is indicative of a pulse curve of the user, including both systolic and diastolic pressure, as well as other information (e.g., heart rate). As described above, each LED 150 is a near-infrared LED, which tends to transmit deeper through the skin as compared to green light generated by green LEDs. Thus, the PPG sensor 148 may measure deeper arterial vessels as compared to systems that use green light. Additionally, compared to near-infrared light, green light is heavily absorbed by darker skin tones. Thus, the PPG sensor 148 including near-infrared LED 150 may provide improved pulse measurement for all skin tones, dark to light, as compared to systems that use green light.

In block 1106 the processor 120 collects sensor data from the bioimpedance sensor 154. As described above, the electrode 156 in contact with the user's skin is used to measure changes in impedance due to changes in cellular shapes and orientations, which occur as a pulse wave moves through the arteries adjacent to the cells. Accordingly, the sensor data collected by the bioimpedance sensor 154 is also indicative of the pulse curve of the user. The sensor data collected from the PPG sensor 148 and the bioimpedance sensor 154 thus represent the pulse curve of the user taken simultaneously from the same location on the user's body but relying on different anatomical mechanics.

In block 1108 the processor 120 may collect sensor data from one or more of the context sensors 136. Such sensor data may measure features of the environment and/or the user other than the pulse curve, which may contribute noise components to the sensor data measured by the multimodal sensors 146. Additionally, this sensor data may be used to determine one or more mechanical variables (e.g., applied pressure or temperature), which may be used for accurate determination of biometric data. Still additionally, in some embodiments this context sensor 136 data may be used to provide contextual information regarding the location of the patient's arm (or other location of the wearable device 100) relative to the heart and the patient's activity (e.g., active exercise, rest, etc.).

In some embodiments, in block 1110 the processor 120 collects sensor data from the accelerometer 138. In some embodiments, in block 1112 the processor 120 collects sensor data from the gyroscope 140. Such motion sensor data may be used to measure macro-motions due to physical activity or micro-motions due to the breathing or small sensor movements relative to the heterogeneous tissue. In some embodiments, in block 1114 the processor 120 collects sensor data from the temperature sensor 142. In some embodiments, in block 1116 the processor 120 collects sensor data from the pressure sensor 144. Pressure sensor 144 data may be used to correct or otherwise calibrate multimodal sensor data received from the multimodal sensors 146, for example by determining the value of one or more mechanical variables.

In block 1118 the processor 120 corrects the multimodal sensor data from the multimodal sensors 146 based on sensor data from the contextual sensors 136. As described above, the multimodal sensor data includes data received from both the PPG sensor 148 and the bioimpedance sensor 154. Sensor data from the PPG sensor 148 and the bioimpedance sensor 154 may be correlated or otherwise combined to generate multimodal sensor data. Due to redundancy involved in using two different sensing modalities to measure the pulse curve at the same location, the multimodal sensor data may already have reduced noise. The contextual sensor 136 data may be used to further reduce noise in the multimodal sensor data. For example, motion sensor data from the accelerometer 138 and/or the gyroscope 140 may be used to remove motion artifacts or other noise components caused by macro-motion or micro-motion from the multimodal sensor data.

In block 1120 the processor 120 determines blood pressure data based on the corrected multimodal sensor data. The blood pressure data may include systolic pressure, diastolic pressure, or other blood pressure data. Of course, although illustrated as determining blood pressure data, it should be understood that in some embodiments the processor 120 may determine other biometric data based on the corrected multimodal sensor data, such as heart rate, heart rate variability, cardiac output, respiratory signature, or other biometric data.

In some embodiments, in block 1122 the processor 120 may determine pulse transit time (PTT) and/or pulse wave velocity (PWV) based on the corrected multimodal sensor data. In an embodiment, the processor 120 may collect multimodal sensor data from two or more multimodal sensors 146 positioned on the wearable device 100, such as from the sensors 146 of the wearable device shown in FIGS. 3 and 4. By comparing the pulse curves measured by each of those sensors 146, the processor 120 may determine a different in pulse arrival time at each sensor 146. That is, the processor 120 may determine when a particular pulse wave was measured by each sensor 146 and determine the time difference between those measurements. This time difference may be relatively small (e.g., on the order of about 0.1 ms), and thus the sampling rate for the multimodal sensors 146 may be above 10 kHz. This measured different in pulse arrival time is the pulse transit time (PTT) for the pulse to travel the length of the wearable device 100 between the multimodal sensors 146, which may be a few centimeters or other predetermined length. Using this measured PTT and that predetermined length, the processor 120 may determine pulse wave velocity (PWV).

In some embodiments, in block 1124 the processor 120 may determine one or more additional features based on the pulse curve signals from the PPG sensor 148 and the bioimpedance sensor 154. For example, the processor 120 may determine one or more changes in wave shape, amplitude, frequency, slope, area under the curve, key points along the pulse curve (e.g., peaks, notches, inflection points, or other points), and derivatives of the pulse curve.

In block 1126, the processor 120 records or transmits the blood pressure data. The blood pressure data may be recorded locally, for example to the data storage device 126, for later review. Additionally or alternatively, the blood pressure data may be transmitted to a remote device using the communication circuitry 128. For example, the blood pressure data may be transmitted to a paired smartphone using a Bluetooth low energy protocol, the blood pressure data may be transmitted to a laptop or desktop computer for further processing, or to another remote device. After recording or transmitting the blood pressure data, the method 1100 loops back to block 1102 to continue collecting sensor data and determining blood pressure data.

1. A wearable device comprising:
a flexible substrate;
a multimodal sensor coupled to the flexible substrate, wherein the multimodal sensor comprises a photoplethysmography sensor and a bioimpedance sensor.

2. The wearable device of claim 1, further comprising a processor coupled to the flexible substrate, wherein the processor is configured to receive sensor data from the multimodal sensor.

3. The wearable device of claim 1, wherein the bioimpedance sensor comprises an electrode positioned on an external surface of the wearable device.

4. The wearable device of claim 3, wherein the photoplethysmography sensor comprises a light-emitting diode (LED) and a photodiode.

5. The wearable device of claim 4, wherein the LED comprises a near-infrared LED.

6. The wearable device of claim 4, wherein the LED and the photodiode are positioned adjacent to the electrode on the external surface of the wearable device.

7. The wearable device of claim 1, further comprising a second sensor configured to generate sensor data indicative of an environment of the wearable device.

8. The wearable device of claim 7, wherein the second sensor comprises a temperature sensor or a pressure sensor.

9. The wearable device of claim 1, further comprising a second sensor configured to generate sensor data indicative of motion of the wearable device.

10. The wearable device of claim 9, wherein the second sensor comprises an accelerometer or a gyroscope.

11. The wearable device of claim 1, further comprising a wireless communication circuit.

12. The wearable device of claim 1, wherein the flexible substrate comprises a first side and a second side opposite the first side, wherein the multimodal sensor is positioned on the first side of the wearable device, and wherein the wearable device further comprises an adhesive coating coupled to the first side, wherein the adhesive coating surrounds the multimodal sensor.

13. The wearable device of claim 1, further comprising a processor configured to receive sensor data from the photoplethysmography sensor of the multimodal sensor and the bioimpedance sensor of the multimodal sensor.

14. The wearable device of claim 13, further comprising a flexible conductive path coupled to the flexible substrate, wherein the flexible conductive path electrically couples the processor to the multimodal sensor.

15. The wearable device of claim 13, wherein the processor is further configured to generate biometric data based on the sensor data received from the photoplethysmography sensor and from the bioimpedance sensor, wherein the biometric data comprises blood pressure data.

16. The wearable device of claim 1, wherein the multimodal sensor further comprises an integrated pressure sensor.

17. The wearable device of claim 16, wherein the multimodal sensor comprises a first sensor frame and a second sensor frame, wherein the first sensor frame is coupled to the flexible substrate, wherein the integrated pressure sensor is coupled between the first sensor frame and the second sensor frame, and wherein the photoplethysmography sensor and the bioimpedance sensor are coupled to the second sensor frame.

18. The wearable device of claim 1, wherein the flexible substrate comprises a stretchable composite.

19. The wearable device of claim 1, wherein the flexible substrate comprises a breathable composite.

20.-33. (canceled)

34. A method for continuous cuffless blood pressure monitoring, the method comprising:
receiving, by a processor, first sensor data from a photoplethysmography sensor of a multimodal sensor device, wherein the multimodal sensor device is included in a wearable device, and wherein the first sensor data is indicative of a pulse curve of a user;
receiving, by the processor, second sensor data from a bioimpedance sensor of the multimodal sensor device, wherein the second sensor data is indicative of the pulse curve of the user; and
determining, by the processor, blood pressure data based on the first sensor data and the second sensor data.

35.-47. (canceled)
